(19) United States
(12) Patent Application Publication
(10) Pub. No.: US 2021/0186360 A1
(43) Pub. Date: Jun. 24, 2021

(54) SMART SCALE SYSTEMS WITH BODY IMAGING AND ASSOCIATED DEVICES AND METHODS

(71) Applicant: Wyze Labs, Inc., Kirkland, WA (US)

(72) Inventors: Dongsheng Song, Bothell, WA (US); Shiwen Niu, Redmond, WA (US)

(21) Appl. No.: 17/131,176

(22) Filed: Dec. 22, 2020

Related U.S. Application Data
(60) Provisional application No. 62/952,972, filed on Dec. 23, 2019.

(51) Int. Cl. A61B 5/0537; A61B 5/00

(52) U.S. Cl. CPC A61B 5/0537; A61B 5/6898; A61B 5/0022; A61B 5/742

(57) ABSTRACT

Smart scale systems with body imaging and associated devices and methods are disclosed herein. In some embodiments, a user device may acquire biometric information generated from a network-accessible scale and image information from an imaging device. Received biometric information and media information associated with a user may be associated with a timestamp indicative of a time that the information was captured. A series of captured biometric information and media information for the user may be chronologically ordered according to the timestamps, which may allow for generation a video or interactive dashboard showing a time-lapsed display of the biometric information and media information of the user during a body transformation journey.

### CROSS-REFERENCE TO RELATED APPLICATION(S)

The application claims priority to and the benefit of U.S. Provisional Patent Application No. 62/952,972, filed Dec. 23, 2019, which is incorporated herein by reference in its entirety.

### TECHNICAL FIELD

The present disclosure relates generally to smart scale systems, and more particularly to smart scale systems with techniques for capturing and presenting both body composition measurements and images.

### BACKGROUND

Scales are configured to determine body composition information relating to a user. For example, responsive to a living body stepping on a scale, the scale can determine a weight of the living body. In many cases, scales can include sensors that measure various body composition metrics using various techniques. On such technique can include bioelectrical impedance analysis (BIA). Scales utilizing BIA techniques can measure various detailed body composition measurements using electrical current traveling through the living body.

While scales can capture body composition metrics of the user, simply capturing these metrics may provide incomplete or insufficient information of a body transformation journey. Example body transformation journeys may include a time period during which the user loses weight (e.g., a weight-loss journey) or a time period during which the user develops their musculature (e.g., a body building journey, a rehabilitation journey). During a body transformation journey, a user may have a substantial change to the body composition over a period of time. This change to the body composition of the user may be inadequately depicted simply by captured body composition metrics.

One mechanism for addressing this issue is to implement a scale that can communicate with a user device to record body composition information measured by the scale. For example, an application on a mobile phone of a user can store weight and body mass information relating to the user. However, simply tracking weight information of a user often may not adequately capture the substantial changes to the body composition of the user during a body transformation journey.

### BRIEF DESCRIPTION OF THE DRAWINGS

The present embodiments are illustrated by way of example and are not intended to be limited by the figures of the accompanying drawings.

FIG. 1 is a representative environment within which some embodiments may be implemented.

FIG. 2 is a block diagram of a computing device that may be used to implement the techniques introduced here.

FIG. 3 is an example block diagram of a signaling process illustrating biometric information and image information acquisition in an environment as disclosed herein.

FIG. 4 is a block diagram of a scale configured in accordance with embodiments of the present technology.

FIG. 5 is a flow diagram of a method performed by a network-accessible scale to acquire biometric information in accordance with embodiments of the present technology.

FIG. 6 is an illustration of an environment that includes multiple imaging devices in accordance with embodiments of the present technology.

FIG. 7 is a flow diagram of a method to process biometric information and image information in accordance with embodiments of the present technology.

FIGS. 8A-8B are example user interfaces illustrating an interactive dashboard that includes both biometric information and image information.

FIGS. 9A-9B are example user interfaces illustrating a video that includes a time-lapse of both biometric information and image information.

FIGS. 10A-10C are example block diagrams illustrating a process to activate a device in an environment as described herein.

FIG. 11 is a system diagram illustrating an example of a computing environment in which the present embodiments may be implemented.

Like reference numerals refer to corresponding parts throughout the figures and specification.

### DETAILED DESCRIPTION

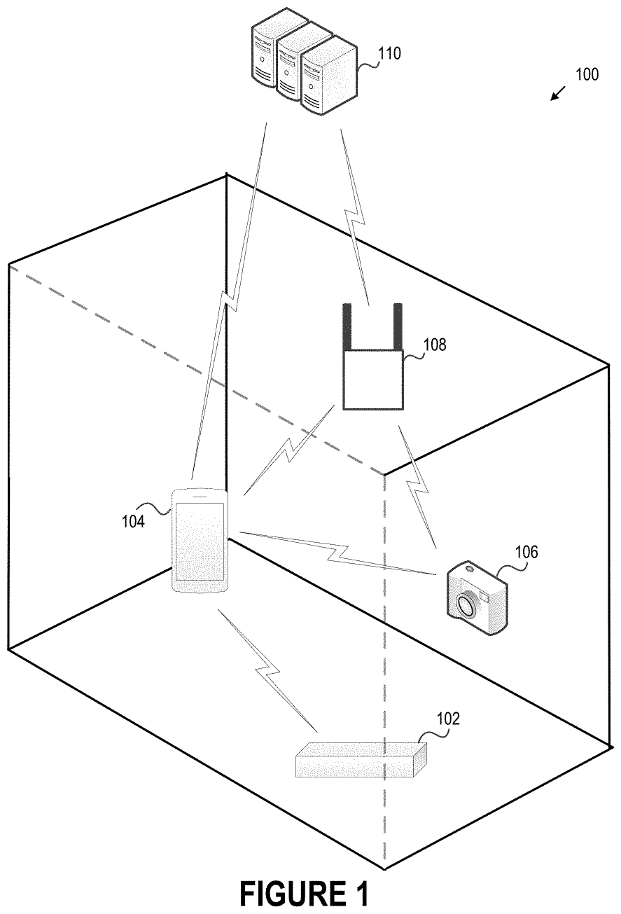

Smart scale systems with body imaging and associated devices and methods are disclosed herein. In some embodiments, for example, a smart scale system can include a scale configured to communicate biometric information of a user to a user device, an imaging device that captures an image of the user's body as the scale measures the biometric data, and an application that displays a dashboard including the captured biometric data and images of the user across a timeline having multiple time points. Specific details of several embodiments of the technology are described below with reference to FIGS. 1-11. Although many of the embodiments are described below with respect to devices, systems, and methods for capturing weight loss and body building journeys, other applications and other embodiments in addition to those described herein are within the scope of the technology. For example, the present technology may be used to track body development over periods of time for other reasons, such as rehabilitation and other medical needs. Additionally, several other embodiments of the technology can have different configurations, components, or procedures than those described herein, and that features of the embodiments shown can be combined with one another. A person of ordinary skill in the art, therefore, will accordingly understand that the technology can have other embodiments with additional elements, or the technology can have other embodiments without several of the features shown and described below with reference to FIGS. 1-11.

Many scales can be configured to send body composition metrics to a user device (e.g., mobile phone), and the user device can record this data over time. However, simply tracking raw data over a period of time may be an insufficient illustration of a user's body composition over time. For example, to supplement tracked body composition information to illustrate one's weight loss journey, a user may attempt to capture and identify images of their body captured during the weight loss journey. This may include accessing various websites or computing devices to search for and identify images during the weight loss journey. When images are identified, the user may compile the images to generate a time-lapse series of images representing the weight loss journey. Such a process may be an insufficient use of computational resources, as various devices may be repeatedly accessed to retrieve images of the user over a network. Further, sending various requests for images among devices in a network and transmitting images across the network may result in an inefficient user of network resources.

Introduced here, accordingly, are systems, devices, and methods for acquiring biometric information of a user along with media information (e.g., images, videos) of the user over a period of time that can be utilized in generating a video or dashboard illustrating changes to a body composition of a user over a period of time. The biometric information and media information associated with a user may be associated with a timestamp indicative of a time that the information was captured. A series of captured biometric information and media information for a user may be chronologically ordered according to the timestamps, which may allow for generation a video or interactive dashboard showing a time-lapsed display of the biometric information and media information of the user during a during a period of time, such as during a specific period of time (e.g., one or more years, one or more months, one or more days), over the course of a weight loss regime, during a muscle building program, during physical therapy, and/or other body transformation journey.

The present embodiments may acquire both biometric information of a user and media information and generate a multi-media presentation of this information with an increased efficiency in utilization of network and computer resources. For instance, rather than having to retrieve images and body composition metrics from a variety of devices over a network, a device (e.g., a network-accessible server system, user device) can acquire and store a series of biometric information and images of a user. A multimedia presentation of chronologically ordered information can be generated with greater efficiency by accessing received information from the device and chronologically ordering the information.

A network-accessible scale (also referred to as a “scale”) may capture a set of biometric information (e.g., weight information, body mass information, BIA-based measurements) associated with the user based on receipt of a triggering event (e.g., receipt of a request message, detecting a user has stepped on the scale). The scale can send the acquired biometric information to a user device (e.g., a mobile phone associated with the user).

The user device can request an imaging device (or simply “camera”) to capture image(s) of the user and send the captured image(s) to the user device. The user device can associate the received biometric information and image(s) with a timestamp indicative of a time/date that the information was captured. In some embodiments, the imaging device can be communicatively coupled to the user device and/or the scale, and can capture image(s) automatically when the scale takes the biometric measurements (either simultaneously or in succession).

A network-accessible server system can receive the biometric information, image(s), and timestamp information from the user device and process the information. The network-accessible server system may maintain multiple sets of biometric information and images of the user captured at various times. Upon a request, the network-accessible server system can generate an interactive dashboard and/or a video that includes the biometric information and images of the user ordered chronologically based on the timestamps.

The generated interactive dashboard and/or video may be illustrative of a user body transformation journey that illustrates a time-lapse depiction of body composition metrics and images of the user. The interactive dashboard and/or video may provide a detailed illustration of changes to the body composition of the user during the course of a body transformation journey. This provides information about not only the biometrics, but also the change of shape and physical appearance of the body over time, such as which portions of the body undergo changes (e.g., decreases in size, increases in size, muscle definition, muscle deterioration) and when. The generated interactive dashboard or video can be viewed, modified, and/or uploaded to various applications/websites that can be shared/accessed by others to share a body transformation journey of the user. As an example, during a weight loss journey, an interactive dashboard may allow for one to view a series of images and metrics illustrating the loss of weight by the user and changes to body composition metrics of the user during the weight loss journey.

In various embodiments, the systems and methods described herein can analyze the image data (e.g., images, videos) to provide additional information to the user. For example, the image data can be analyzed using software programs, including machine learning, that determine one or more parameters related to the image of the user, such as measurements of certain body parts (e.g., waist, hips, thighs, calves, arms), muscle definition, body shape, and/or changes in these parameters over time.

In the following description, numerous specific details are set forth such as examples of specific components, circuits, and processes to provide a thorough understanding of the present disclosure. Also, in the following description and for purposes of explanation, specific nomenclature is set forth to provide a thorough understanding of the present embodiments. However, it will be apparent to one skilled in the art that these specific details may not be required to practice the present embodiments. In other instances, well-known circuits and devices are shown in block diagram form to avoid obscuring the present disclosure.

The term “coupled” as used herein means connected directly to or connected through one or more intervening components or circuits. Any of the signals provided over various buses described herein may be time-multiplexed with other signals and provided over one or more common buses. Additionally, the interconnection between circuit elements or software blocks may be shown as buses or as single signal lines. Each of the buses may alternatively be a single signal line, and each of the single signal lines may alternatively be buses, and a single line or bus might represent any one or more of a myriad of physical or logical mechanisms for communication (e.g., a network) between components. The present embodiments are not to be construed as limited to specific examples described herein but rather to include within their scope all embodiments defined by the appended claims.

### System Overview

FIG. 1 is a representative environment 100 within which some embodiments may be implemented. An example environment 100 may include any of a network-accessible scale 102, a user device 104, an imaging device 106, a wireless network node 108, and a network-accessible server system 110.

The network-accessible scale 102 (or simply “scale 102”) may include a device that can capture biometric information of a living body. Biometric information can include any measurable characteristic of a body composition of a living body. Examples of biometric information that can be measured may include a weight, body mass index (BMI), or Bioelectrical Impedance Analysis (BIA) based information (e.g., body fat, muscle, body water, lean body mass, bone mass, protein, visceral fat, basal metabolic rate (BMR), metabolic age) relating to a living body.

The network-accessible scale 102 may include a plurality of sensors that can be used to determine various biometric information of a user. For example, a sensor can detect the weight of a user stepping on the scale 102. In some embodiments, the network-accessible scale 102 may determine a change in pressure indicative of a user stepping on the scale 102.

The environment 100 may include a user device 104. The user device 104 can include an electronic device (e.g., mobile phone, computer) that can interact with various devices in the environment via one or more communications interfaces. For example, the user device 104 can communicate with the scale 102 via a wireless communication interface (Bluetooth®, Bluetooth® Low Energy (BLE), Near-Field Communication (NFC), Wi-Fi, etc.).

In some embodiments, the user device 104 may be communicatively coupled to a wearable electronic device (or a “smart watch”). The wearable electronic device can provide an input to the user device 104 (e.g., a voice command, selection of a button) that indicates a request to perform a task (e.g., capture weight information by the scale 102 and capture image by imaging device 106).

As an example, the wearable electronic device can send a digitized audio stream representing a voice command providing a request to capture weight information by scale 102 and capture an image by imaging device 106. In this example, the user device 104 can identify the requested task and the devices to perform the requested task by inspecting the digitized audio stream. Accordingly, user device 104 can send a request message to scale 102 and imaging device 106 to capture weight information/image of the user.

The user device 104 may include a mobile application. The mobile application can interact with various devices (e.g., scale 102, camera 106) in the environment 100 as described with greater detail below. The user device/motion application can be associated with a user profile associated with a user. As noted below, the user profile may be utilized in determining whether the scale 102 and/or imaging device 106 will send information to the user device 104.

The environment 100 may include an imaging device 106. The imaging device 106 (or simply “camera”) may include an electronic device configured to capture image(s) of an environment or a user. As noted below, the camera 106 can capture images of the user based on receipt of an input, such as a message from the user device requesting the camera 106 to capture the image.

The environment 100 may include a wireless network node 108. The wireless network node 108 can include a wireless router configured to communicate with devices in a network using a suitable wireless protocol (e.g., Wi-Fi).

The wireless network node 108 can be a network device (e.g., a router, gateway), which in computer networking sense, is a node that is assumed to know how to forward packets on to other networks. In a home or small office environment, the wireless network node 108, such as a digital subscriber line (DSL) router or cable router that connects the local area network (LAN) to the Internet acts as the default gateway for all network devices. For example, the wireless network node 108 and a network (e.g., the internet) may be connected via a twisted pair cabling network, a coax cable network, a telephone network, or any suitable type of connection network. In some embodiments, wireless network node 108 may be connected to a network wirelessly (e.g., which may include employing a data traffic network based on wireless telephony services such as 3G, 3.5G, 4G LTE and the like).

Any devices in the environment 100 can communicate either directly or indirectly, via one or more wireless network communication technologies, such as WLAN (e.g., Wi-Fi), Bluetooth, etc. The IEEE 802.11 standards are a set of WLAN technology specifications commonly seen for implementing wireless local area network (WLAN) computer communication. Examples of different wireless communication protocols in the IEEE 802.11 family of standards can include IEEE 802.11a, IEEE 802.11b, IEEE 802.11n, IEEE 802.11ac, and so forth. For example, a client device (e.g., 102, 104, 106) establishes connection with the wireless network node 108, the wireless network node 108 can communicate the traffic to the outside world (e.g., wide area network (WAN) and/or “the Internet”).

Although not shown for simplicity, the wireless network node 108 may include one or more processors, which may be general-purpose processors or may be application-specific integrated circuitry that provides arithmetic and control functions to implement the techniques disclosed herein on the wireless network node 108. The processor(s) may include a cache memory (not shown for simplicity) as well as other memories (e.g., a main memory, and/or non-volatile memory such as a hard-disk drive or solid-state drive. In some examples, cache memory is implemented using SRAM, main memory is implemented using DRAM, and non-volatile memory is implemented using Flash memory or one or more magnetic disk drives. According to some embodiments, the memories may include one or more memory chips or modules, and the processor(s) on the wireless network node 108 may execute a plurality of instructions or program codes that are stored in its memory.

The environment 100 may include a network-accessible server system 110. The network-accessible server system 110 can include one or more interconnected servers configured to access, store, and process information. In an embodiment, the network-accessible server system 110 can interact with the wireless network node 108 and/or the user device 104.

The client devices (e.g., 102, 104, 106) can connect to and communicate with the wireless network node 108 wirelessly including, for example, using the IEEE 802.11 family of standards (e.g., Wireless LAN), and can include any suitable intervening wireless network devices including, for example, base stations, routers, gateways, hubs, or the like. Depending on the embodiments, the network technology connecting between the client devices and the wireless network node 108 can include other suitable wireless standards such as the well-known Bluetooth communication protocols or near field communication (NFC) protocols. In some embodiments, the network technology between the devices and the wireless network node 108 can include a customized version of WLAN, Bluetooth, or customized versions of other suitable wireless technologies. Client devices can be any suitable computing or mobile devices including, for example, smartphones, tablet computers, laptops, personal digital assistants (PDAs), or the like. Client devices typically include a display and may include suitable input devices (not shown for simplicity) such as a keyboard, a mouse, or a touchpad. In some embodiments, the display may be a touch-sensitive screen that includes input functionalities. Additional examples of the devices can include network-connected cameras (or “IP cameras”), home sensors, and other home appliances (e.g., a “smart refrigerator” that can connect to the Internet).

It is noted that one of ordinary skill in the art will understand that the components of FIG. 1 are just one implementation of the network environment within which present embodiments may be implemented, and the various alternative embodiments are within the scope of the present embodiments. For example, the environment 100 may further include multiple imaging devices 106, intervening devices (e.g., switches, routers, hubs), etc.

Environment 100 as shown in FIG. 1 is representative for illustrative purposes only. The techniques disclosed here can be applicable to alternative implementations that may have different configurations. One or more elements or devices introduced in FIG. 1 may be eliminated, combined, or separated. For example, any of the network-accessible scale 102, user device 104, and network-accessible server system 110 can perform any of the tasks relating to the embodiments as described herein.

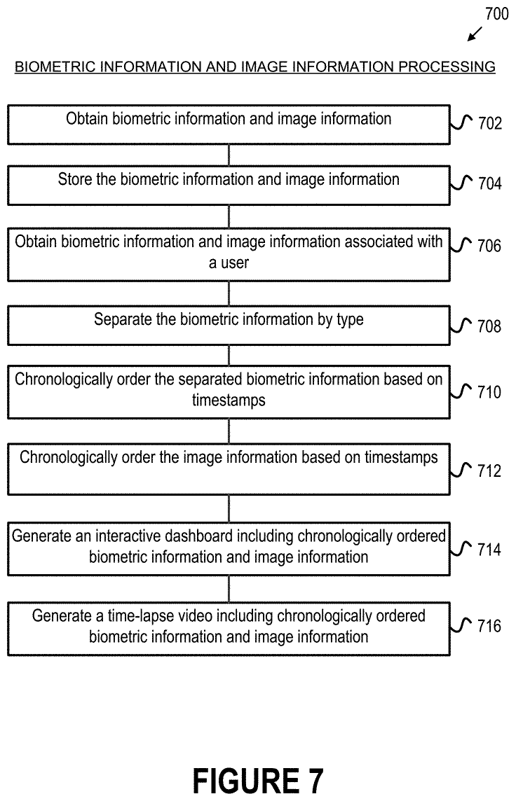

In one example, the network-accessible scale 102 can perform any of the steps as described with reference to FIG. 7.

With the environment introduced above in mind, various techniques for capturing both biometric information and imaging information are described in more detail below, with continued reference to the elements in FIG. 1.

Device Architecture

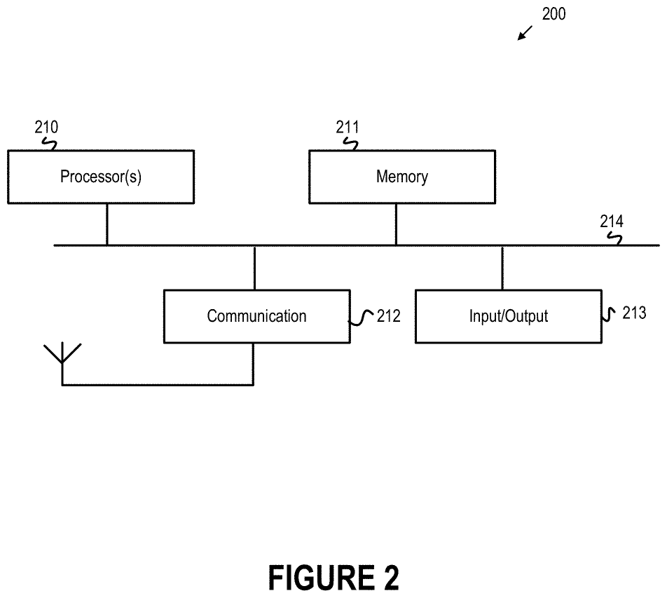

FIG. 2 is a high-level block diagram showing an example of a computing device 200 that can be used to implement one or more devices (e.g., scale 102, user device 104, camera 106, wireless network node 108, network-accessible server system 110) introduced here.

In the illustrated embodiment, the computing system 200 includes one or more processors 210, memory 211, a communication device 212, and one or more input/output (I/O) devices 213, all coupled to each other through an interconnect 214. The interconnect 214 may be or may include one or more conductive traces, buses, point-to-point connections, controllers, adapters and/or other conventional connection devices. The processor(s) 210 may be or may include, for example, one or more general-purpose programmable microprocessors, microcontrollers, application specific integrated circuits (ASICs), programmable gate arrays, or the like, or a combination of such devices. The processor(s) 210 control the overall operation of the computing device 200. Memory 211 may be or may include one or more physical storage devices, which may be in the form of random access memory (RAM), read-only memory (ROM) (which may be erasable and programmable), flash memory, miniature hard disk drive, or other suitable type of storage device, or a combination of such devices. Memory 211 may store data and instructions that configure the processor(s) 210 to execute operations in accordance with the techniques described above. The communication device 212 may be or may include, for example, an Ethernet adapter, cable modem, Wi-Fi adapter, cellular transceiver, Bluetooth transceiver, or the like. Depending on the specific nature and purpose of the processing device 200, the I/O devices 213 can include devices such as a display (which may be a touch screen display), audio speaker, keyboard, mouse or other pointing device, microphone, camera, etc.

Biometric Information and Image Information Acquisition

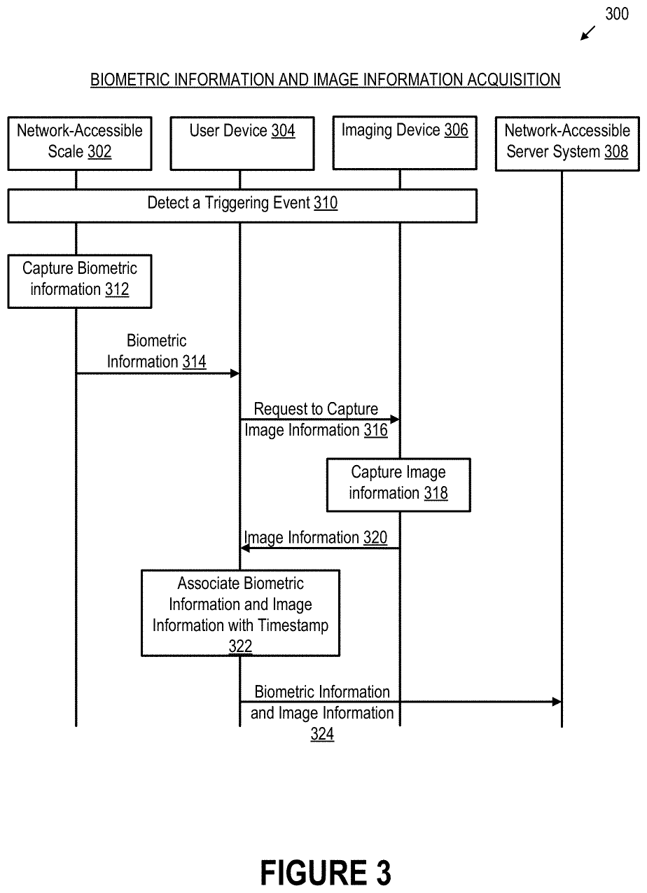

FIG. 3 is an example block diagram of a signaling process 300 illustrating biometric information and image information acquisition in an environment as disclosed herein. As shown in FIG. 3, the signaling process 300 can include information acquisition and transmission between a network-accessible scale, a user device 304, an imaging device 306, and a network-accessible server system 308.

Any of the network-accessible scale 302, user device 304, and/or imaging device 306 may detect a triggering event (block 310). A triggering event may include an occurrence that initiates the capture of biometric information and/or image information of a user. Example triggering events include the scale 302 determining that a user has stepped on the scale 302, the user device 304 coming within a wireless communication range of the scale 302, the user device 304 sending a message to the scale 302 and/or imaging device 306, the user device 304 receiving an indication on a mobile application from a user, the imaging device 306 detecting (e.g., using motion sensing, facial recognition) that a user is near the scale 302 or has stepped on the scale 302, etc.

The scale 302 can capture biometric information (block 312). This may include obtaining sensor data received from a plurality of sensors and processing the sensor data to derive biometric information, which may be referred to as “body composition metrics.” The scale 302 can determine various types of body composition metrics, such as weight (in pounds), body fat (as a percentage), lean body mass (in kilograms), BIA-based measurements, etc.

The scale 302 can send the captured biometric information to the user device 304 (block 314). The biometric information can be sent via a communication interface. The communication interface may include a short-range wireless communication interface, such as Bluetooth®, Bluetooth® low energy (BLE), NFC, etc.

The user device 304 can send a request to capture image information to the imaging device 306 (block 316). This request can instruct an imaging device 306 to capture image information of a user. The request may be sent responsive to receiving the biometric information, as described in block 314.

In some embodiments, request can be transmitted via a wireless communication interface different than that of the communication interface. For example, the communication interface between the user device 304 and scale 302 can be a Bluetooth® interface and the wireless communication interface between the user device 304 and imaging device 306 can include a Wi-Fi interface. The communication interface between the user device 304 and imaging device 306 can include a wireless communication range (e.g., the maximum distance that communicating devices can successfully transmit data) greater than a wireless communication range between the user device 304 and scale 302.

The imaging device 306 can be configured to capture image information (block 318). Imaging device 306 (e.g., cameras) can capture image(s) of the user responsive to either detecting a triggering event (block 310) or receiving a request to capture image information (block 318). As discussed in greater detail below, multiple cameras 306 can capture images of the user from various perspectives, which may be utilized in generating a 3-dimensional image of the user.

The imaging device 306 may send captured image information to the user device 304 (block 320). In some embodiments, the imaging device 306 may send the image to the network-accessible server system 308 via a wireless network node (e.g., wireless network node 108).

The user device 304 may associate the received biometric information and the received image information with a timestamp (block 322). The timestamp can include a digital record of a time of an occurrence of an event. Example events that can identified in the timestamp can include any of the time that the triggering event was detected, the time that the biometric information and/or the image information was received at the user device, the time that the biometric information and/or the image information was sent to the network-accessible server system, etc. As detailed below, timestamps may be utilized in ordering a series of biometric information and images acquired at various times.

The user device 304 may send the biometric information and the image information to the network-accessible server system 308 (block 324). In response to receiving the biometric information and the image information, the network-accessible server system 308 may perform a further action (e.g., store the information, process the information), as described in greater detail below.

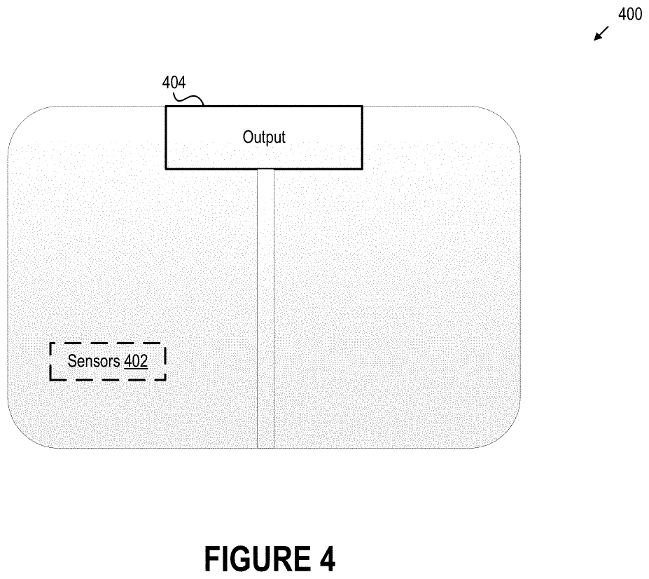

FIG. 4 is an example block diagram of a scale 400. As noted above, the scale 400 can include a network-accessible device configured to determine various body composition metrics relating to a user.

The scale 400 may include a plurality of sensors 402. The plurality of sensors 402 located within the scale 400 may be configured to capture information that can be used to determine various body composition measurements of a user.

An example sensor 402 may include a weight sensor that can measure the weight of a user stepping on the scale 400. Another example sensor 402 can include a bioelectrical impedance analysis (BIA) sensor that utilizes electric current (e.g., using electrodes) flowing through the body of the user to calculate impedance of the body. BIA sensors can detect various compositions of body water, body mass, body fat, etc. of the user. Examples of biometric information that can be measured may include a weight, body mass index (BMI), or Bioelectrical Impedance Analysis (BIA) based information (e.g., body fat, muscle, body water, lean body mass, bone mass, protein, visceral fat, basal metabolic rate (BMR), metabolic age) relating to a living body.

In some embodiments, the sensors 402 can include one or more imaging devices (e.g., cameras) integrated in the scale 400 to capture images of the user. The imaging devices integrated in the scale 400 may perform any of the functionality of the imaging devices (e.g., imaging device 106) as described herein.

The scale 400 may include an output 404 that can display various information to the user. For example, the output 404 can display a date, time, and the weight of the user. In some embodiments, the output 404 can provide indications of various events, such as detection of a user device or that the scale determined a user profile based on generated body composition measurements measured by the scale 400. The output 404 can output any suitable information to improve user experience in interacting with the scale 400.

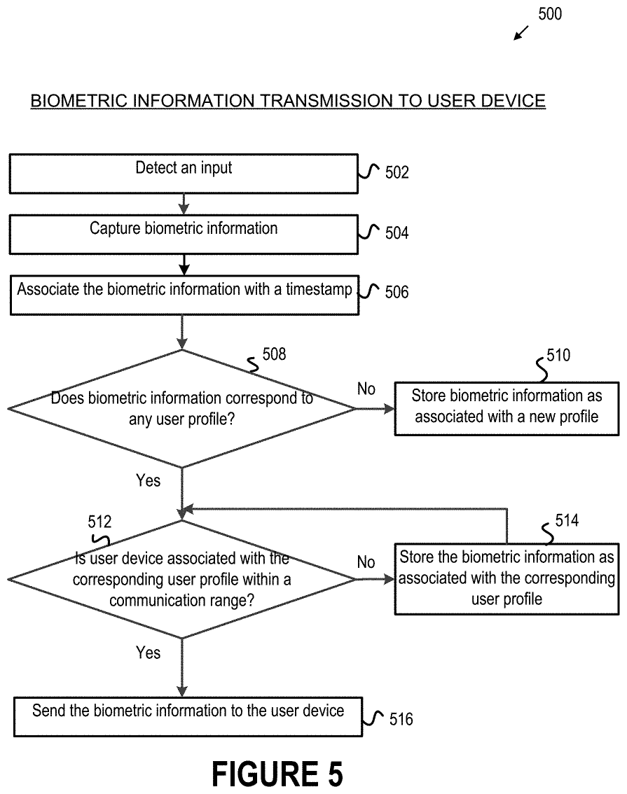

FIG. 5 is an example flow diagram of a method 500 performed by a network-accessible scale to acquire biometric information. The method may include the scale detecting an input (block 502). The input may be indicative of a triggering event, such as the scale detecting that a living body has stepped onto the network-accessible scale, the scale receiving an indication from a user device to capture biometric information, or the scale receiving an indication from a mobile application on a user device indicating that the device is within a communication range of the scale, for example.

The method may include capturing biometric information of a user (block 504). The scale may capture biometric information of a user and determine body composition metrics using sensors included in the scale as described herein.

The method may include associating the biometric information with a timestamp (block 506). The timestamp may be indicative of a time that the biometric information was captured or a time that the input was detected, for example.

The method may include determining whether the captured biometric information corresponds to a user profile (decision block 508). This may include comparing the biometric information with information relating to known user profiles stored in the network-accessible scale to determine whether information relating to any profile is within a threshold similarity to the set of biometric information. The threshold similarity may be a predetermined value or percentage that represents the maximum difference that the biometric information can be from information relating to user profiles to correspond to a user profile.

As an example, a first user profile may have an average weight of 225 pounds and 18% body fat. Additionally, a second user profile may have an average weight of 125 pounds and 12% body fat. If the scale determines that the weight of the user on the scale is 128 pounds with a 12.5% body fat percentage, the scale may determine that the first user profile is outside the threshold similarity (e.g., within 3% of the captured biometric information), while the second user profile is within the threshold similarity to the captured biometric information. Accordingly, in this example, the captured biometric information may be associated with the second user profile.

In some embodiments, the scale can include one or more user profiles associated with users. In these embodiments, the scale can inspect captured body composition information to derive whether a person stepping on the scale corresponds to any of the stored user profiles. The scale can determine that captured body composition information is associated with a new user based on determining that no user profile is within a threshold similarity of the captured body composition information. In other embodiments, a user device (e.g., mobile phone) or a network-accessible server system may include the user profiles and determine whether captured body composition information corresponds to any user profile.

If it is determined that captured biometric information does not correspond to any user profile, the method may include storing the biometric information as associated with a new user profile (block 510). The stored biometric information associated with the new user profile may be sent to any of the user device or a network-accessible server system for further processing.

If it is determined that captured biometric information corresponds to a user profile, the method may include determining whether the user device associated with the corresponding user profile within a communication range (decision block 512). This may include receiving an indication from the user device that the user device is within a wireless communication range of the network-accessible scale.

If the user device associated with the corresponding user profile not within a communication range of the scale, the method may include storing the biometric information as associated with the corresponding user profile (block 514). The scale may repeatedly attempt to determine whether the whether the user device associated with the corresponding user profile within a communication range.

If it is determined that user device associated with the corresponding user profile is within a communication range of the scale, the method may include sending the biometric information to the user device associated with the corresponding user profile (block 516).

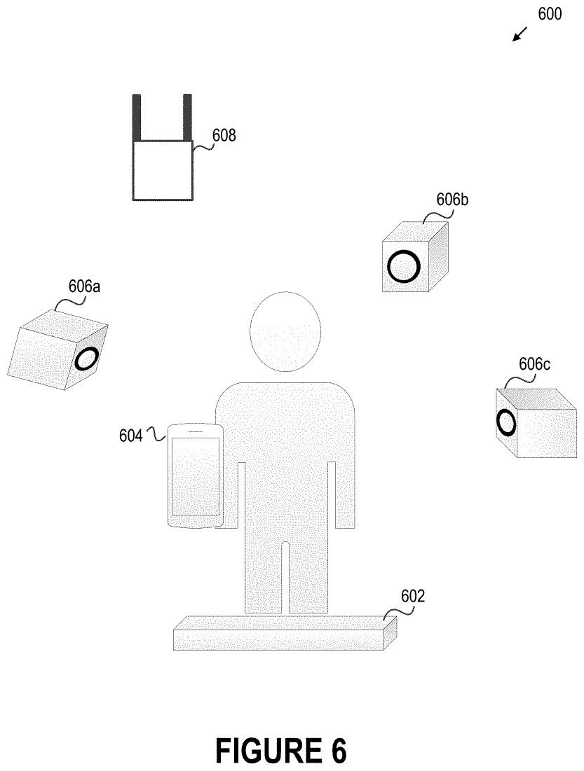

FIG. 6 is an example illustration of an environment 600 that includes multiple imaging devices 606a-c. As shown in FIG. 6, the environment can include a number of imaging devices (e.g., three cameras 606a-c) that can capture images from various perspectives.

Capturing multiple images of a user from multiple perspectives can be used in generating a multi-dimensional image of the user, such as a 3-dimensional image of the user. The images may be stitched together to capture a perspective view or 3-dimensional image of the user that can show multiple angles or perspectives of the user. The cameras 606a-c may be interconnected devices (e.g., internet of things (IoT) devices) in a network that can forward the captured images to any of the user device 604 or a wireless network node 608.

Biometric Information and Image Information Processing

A computing device (e.g., network-accessible server system) may be configured to perform various processing tasks relating to acquired biometric information and image information. FIG. 7 is an example flow diagram of a method 700 to process biometric information and image information.

The method may include obtaining biometric information and image information (block 702). The biometric information and/or image information may be obtained responsive to an input indicative of a triggering event. The triggering event may include any of a network-accessible scale detecting a living body stepping on the network-accessible scale and a mobile device associated with a user being located within a wireless communication range of a network-accessible scale.

In some embodiments, the biometric information is received from a mobile phone via a first communication interface, the mobile phone may receive the biometric information from the network-accessible scale via a second communication interface, the first communication interface including a greater wireless communication range than the second communication interface. The imaging information may be received from a network node via a third communication interface, the network node configured to receive the imaging information from the imaging device via a fourth communication interface.

The method may include storing the received biometric information and image information (block 704). The information may be stored in a first portion of memory associated with a user. The information may be store with a timestamp indicative of a time that the biometric information and the imaging information was captured.

In some embodiments, the received biometric information and/or the received imaging information may be compared with information relating to known user profiles to determine whether information relating to any known user profile is within a threshold similarity to the received biometric information and/or the received imaging information. It may be determined that information relating to the first user profile is within the threshold similarity to the received biometric information and/or the received imaging information. Based on this determination, the biometric information and/or the received imaging information may be stored in a portion of memory associated with the first user profile.

In some embodiments, upon determining that no information relating to any known user profile is within the threshold similarity to the received biometric information and/or the received imaging information, the biometric information, the imaging information, and the timestamp may be stored in a second portion of memory associated with a new user profile.

The method may include obtaining biometric information and image information associated with a user (block 706). This may be performed based on receiving an indication to retrieve information associated with a user that includes timestamps during a time duration. For example, a request may provide an indication to retrieve biometric data and image data for a first user from the last three months.

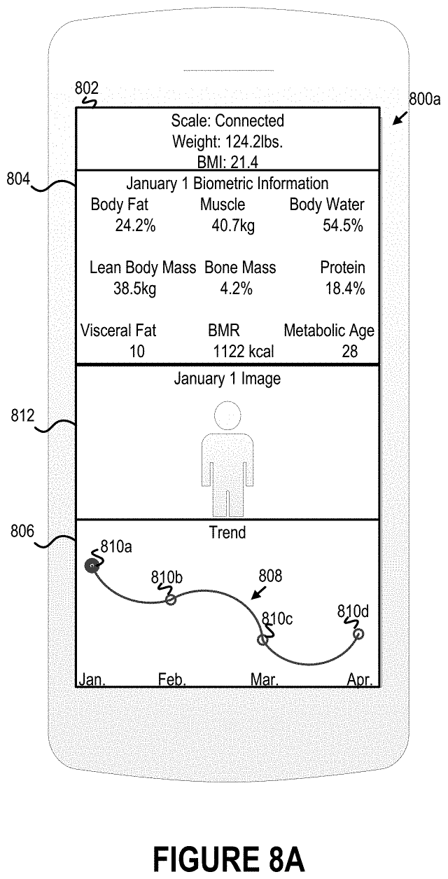

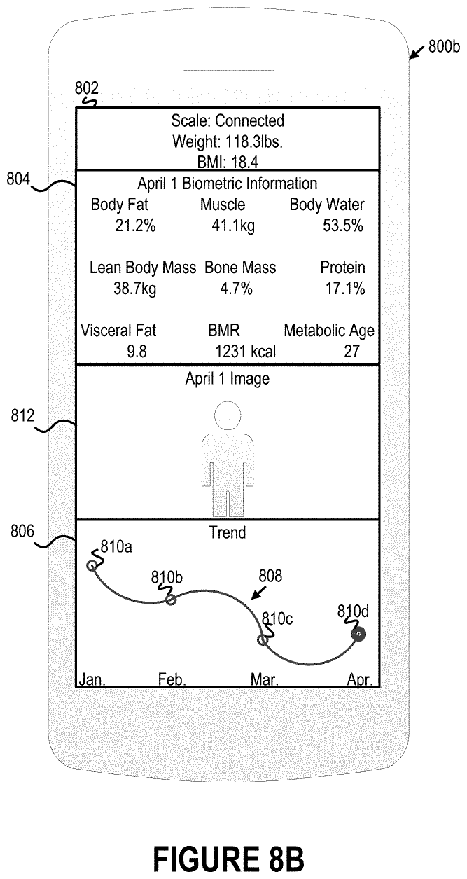

The method may include generating an interactive dashboard (block 708). An interactive dashboard may allow for a user to interact with and view various images and biometric information on the dashboard. An example interactive dashboard is described with respect to FIG. 8.

Generating an interactive dashboard may include separating the biometric information by type (block 708). This may include determining a type of biometric information (e.g., weight, body mass) and separating the data based on type. For example, all biometric information associated with the weight of the user may be identified and separated from other biometric information. Separating the biometric information by type may be utilized in ordering the information and determining trends in the changes in the types of biometric information.

Generating an interactive dashboard may include chronologically ordering the separated biometric information based on timestamps (block 710). This may include inspecting all timestamps and ordering the information based on times indicated by the timestamps. For example, a first set of biometric information includes a timestamp indicating 4:00 pm on January 1. In this example, a second set of biometric information includes a timestamp indicating 7:00 am on January 8. The timestamps can be inspected to order the first set of biometric information ahead of the second set of biometric information.

Generating an interactive dashboard may include chronologically ordering the image information based on the timestamps (block 712). This may include inspecting all timestamps and ordering the information based on times indicated by the timestamps similar to that with respect to ordering the separated biometric information.

The method may include generating an interactive dashboard that includes the ordered biometric information and image information (block 714). This may include displaying a graphical representation of the one or more chronologically ordered biometric information types, such as by a trendline as shown in FIG. 8. The dashboard may also include the series of images ordered chronologically. The dashboard is discussed in greater detail with respect to FIG. 8.

The method may include generating a video that includes a time-lapse of biometric information and image information according to the timestamps (block 716). Generating the video may include chronologically ordering the imaging information according to the timestamps and generating a graphical representation of biometric information types included in the biometric information ordered chronologically according to the timestamps. The video may include the ordered imaging information adjacent to the graphical representation of biometric information types.

Any of the interactive dashboard and the video may include data representing a plurality of measured pieces of biometric information of the user, along with captured images of the user, across a timeline having multiple time points based on a plurality of stored timestamps.

A generated video may be sent to an external device as specified in the request for the video that includes both biometric information and imaging information captured during the second time period.

### Interactive Dashboard and Video Generation

FIGS. 8A-8B are example user interfaces illustrating an interactive dashboard 800 that includes both biometric information and image information. The dashboard 800a-b may be outputted onto a device, such as a mobile device or computer, for example. In some embodiments, the interactive dashboard 800a-b may be displayed via an application executing on a device.

As shown in FIG. 8A, the dashboard 800a can include various information, such as a set of initial information 802, a set of body composition information 804, trendline(s) 806, and image information 812.

The set of initial information 802 displayed on the dashboard may include various information relating to the user, such as the weight of BMI of the user, for example. In some embodiments, general information, such as a current time or an indication that a scale is connected, may be included in the set of initial information 802.

The set of body composition information 804 may include various measurements relating to a body composition of a user at a given time. Example body composition measurements may include body fat, muscle, body water, lean body mass, bone mass, protein, visceral fat, BMR, metabolic age, etc.

As shown in FIG. 8A, the body composition measurements can be associated with a first time period (e.g., January 1). A user may interact with the dashboard to view images and biometric information at various times (e.g., April 1). In some embodiments, the user may select to view the images and biometric information chronologically (e.g., from January 1 to April 1).

The trendline(s) 806 may provide a graphical representation of a type of biometric information as a function of time. For example, a weight of a user can be shown as a trendline using points (e.g., points 810a-d) indicative of the changes in the user's weight over a period of time. A trendline 808 can be added between points 810a-d to illustrate the changes to the weight over time. In some embodiments, selecting a body composition measurement (e.g., set of body composition information 804) may cause the dashboard to display a trendline 808 of the selected body composition measurement.

FIG. 8B illustrates an example interactive dashboard at a second time. For example, while FIG. 8A illustrates information relating to a first date (e.g., January 1), FIG. 8B may illustrate information relating to a second date (e.g., April 1). The differences between the dates can reflect changes to a user body composition and images during the time between the dates.

In the example as shown in FIG. 8B, the dashboard can include a separate set of body composition information 804 and an image of the user captured on April 1 812. The differences in the body composition information and the images of the user in the dashboard can provide a multi-media visualization of a user's body transformation journey.

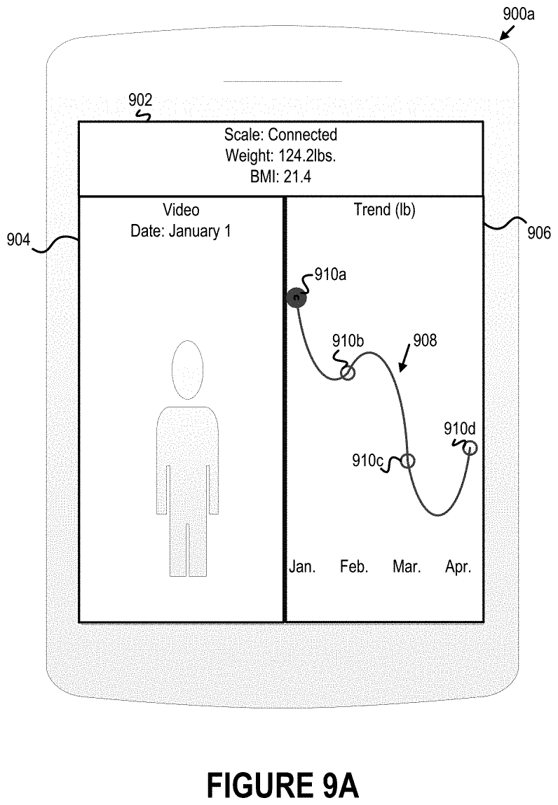

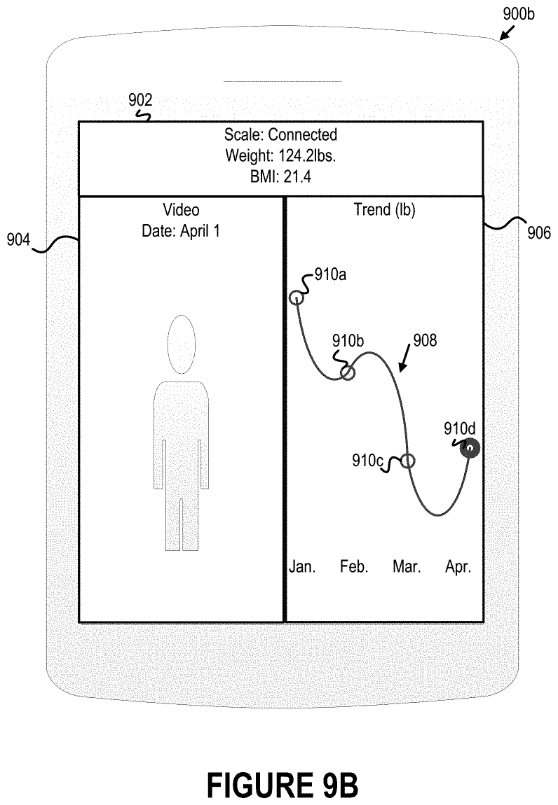

FIGS. 9A-9B are example user interfaces illustrating a video that includes a time-lapse of both biometric information and image information. As noted above, the biometric information and the images of a user can be chronologically ordered and presented as a time-lapsed representation of a user's body transformation journey. In the embodiments as shown in FIGS. 9A-9B, a video 900 depicting both images of the user and a graphical representation of the biometric information may be played on a user device (e.g., mobile phone, tablet, laptop). In some embodiments, the video may be downloaded and viewed by accessing an internet-accessible server that is hosting the video.

As shown in FIG. 9A, the video at a first time (e.g., January 1) may include a banner 902, a first portion of video 904 depicting an image of the user and a second portion of video 906 depicting a graphical representation of biometric information (e.g., a trendline of a user's weight during a timespan). In some embodiments, the first portion of video 904 may include a 3-dimensional image of the user, where as the video plays, the 3-dimensional image of the user may allow for multiple perspectives of the user.

The second portion of video 906 may include one or more trendlines (e.g., trendline 908) of a type of biometric information (e.g., weight of the user, BMI of the user) during a time span (e.g., from January 1 to April 1). In some embodiments, the second portion of video 906 can include a plurality of body composition metrics relating to a first time (e.g., January 1).

FIG. 9B is an illustration of an example video at a second time (e.g., April 1). The video may display a series of images of the user and the biometric information for the user in a chronological order. In an example, the video may begin with a first frame relating to a first time (e.g., January 1) as shown in FIG. 9A and end with a final frame relating to a second time (e.g., April 1) as shown in FIG. 9B.

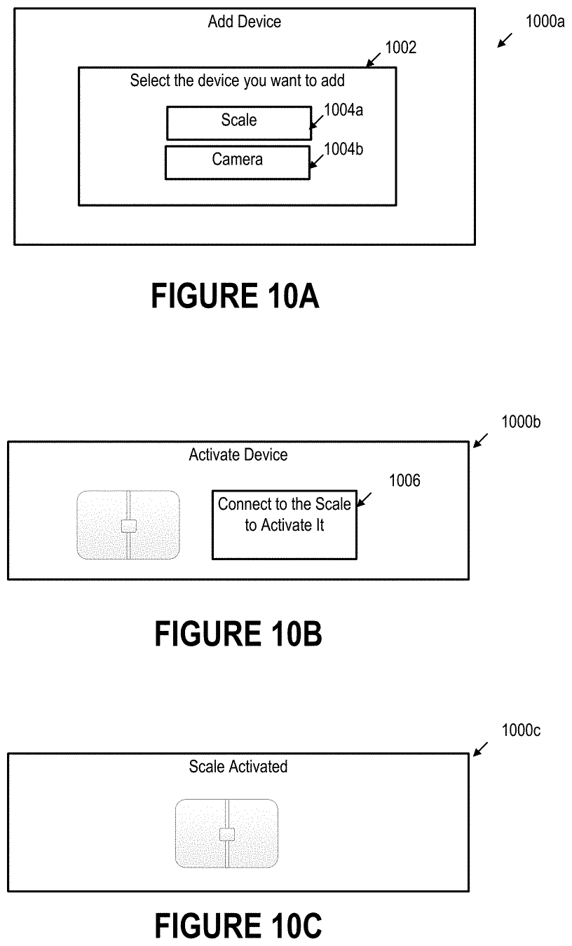

In many cases, a device (e.g., a scale 102, camera 106) may be added to the environment. For example, in order to capture a 3-dimensional image of a user, multiple cameras may be added to the environment. FIGS. 10A-10C are example block diagrams of interfaces that implement a process to activate a device in an environment as described herein.

### Device Activation

A network device can be activated and associated with a user profile. As shown in FIG. 10A, an interface 1000a may allow for a new device to be added into the environment. A device may be added into the environment to assist in performing the steps as described herein. For example, a camera may be added to the environment to capture an image of the user. Adding a device to the environment may associate the device to one or more users such that data acquired from that device can be associated with a user. In the example as shown in FIG. 10A, the interface 1000a may provide a display 1002 requesting a selection of a device (e.g., scale 1004a, camera 1004b) to add.

Upon selection of a device (e.g., scale 1004a), the interface 1000b may indicate an indication to connect a device 1006 (e.g., scale 1004a) to a user device. This request can allow for the user device to connect to the scale via a suitable communication interface (e.g., Bluetooth®). In some embodiments, the user device may initiate a setup procedure with the scale to associate the scale with a user or mobile application.

Upon successful connection to a device (e.g., scale), the interface 1000c can provide a confirmation message indicating that the device is successfully connected. This process may be repeated for any new devices that are to be connected in the environment.

### Example Computing Environment

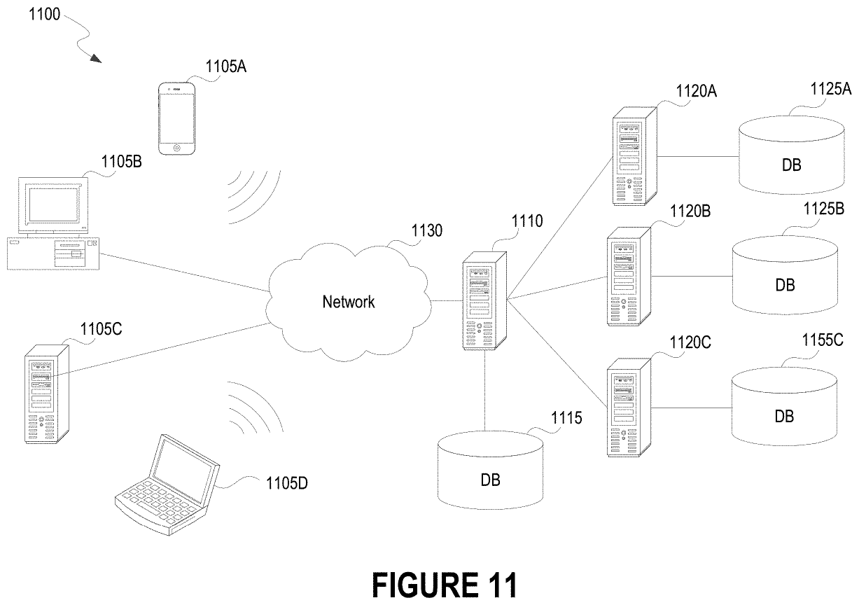

FIG. 11 is a system diagram illustrating an example of a computing environment 1100 in which the present embodiments may be implemented. In some implementations, environment 1100 includes one or more client computing devices 1105A-D. Client computing devices 1105 operate in a networked environment using logical connections 1110 through network 1130 to one or more remote computers, such as a server computing device.

In some implementations, server 1110 is an edge server which receives client requests and coordinates fulfillment of those requests through other servers, such as servers 1120A-C. In some implementations, server computing devices 1110 and 1120 comprise computing systems, such as computer system 100. Though each server computing device 1110 and 1120 is displayed logically as a single server, server computing devices can each be a distributed computing environment encompassing multiple computing devices located at the same or at geographically disparate physical locations. In some implementations, each server 1120 corresponds to a group of servers.

Client computing devices 1105 and server computing devices 1110 and 1120 can each act as a server or client to other server/client devices. In some implementations, servers (1110, 1120A-C) connect to a corresponding database (1115, 1125A-C). As discussed above, each server 1120 can correspond to a group of servers, and each of these servers can share a database or can have its own database. Databases 1115 and 1125 warehouse (e.g., store) information such as user data (e.g., user identifiers, user profiles, etc.), web page data, machine learning models, performance parameters, and so on. Though databases 1115 and 1125 are displayed logically as single units, databases 1115 and 1125 can each be a distributed computing environment encompassing multiple computing devices, can be located within their corresponding server, or can be located at the same or at geographically disparate physical locations.

Network 1130 can be a local area network (LAN) or a wide area network (WAN), but can also be other wired or wireless networks. In some implementations, network 1130 is the Internet or some other public or private network. Client computing devices 1105 are connected to network 1130 through a network interface, such as by wired or wireless communication. While the connections between server 1110 and servers 1120 are shown as separate connections, these connections can be any kind of local, wide area, wired, or wireless network, including network 1130 or a separate public or private network.

### CONCLUSION

Unless contrary to physical possibility, it is envisioned that (i) the methods/steps described above may be performed in any sequence and/or in any combination, and that (ii) the components of respective embodiments may be combined in any manner.

The techniques introduced above can be implemented by programmable circuitry programmed/configured by software and/or firmware, or entirely by special-purpose circuitry, or by a combination of such forms. Such special-purpose circuitry (if any) can be in the form of, for example, one or more application-specific integrated circuits (ASICs), programmable logic devices (PLDs), field-programmable gate arrays (FPGAs), etc.

Software or firmware to implement the techniques introduced here may be stored on a machine-readable storage medium and may be executed by one or more general-purpose or special-purpose programmable microprocessors. A “machine-readable medium”, as the term is used herein, includes any mechanism that can store information in a form accessible by a machine (a machine may be, for example, a computer, network device, cellular phone, personal digital assistant (PDA), manufacturing tool, any device with one or more processors, etc.). For example, a machine-accessible medium can include recordable/non-recordable media (e.g., read-only memory (ROM), random access memory (RAM), magnetic disk storage media, optical storage media, flash memory devices, etc.).

Any of the steps as described in any methods or flow processes herein can be performed in any order to the extent the steps in the methods or flow processes remain logical.

Note that any and all of the embodiments described above can be combined with each other, except to the extent that it may be stated otherwise above or to the extent that any such embodiments might be mutually exclusive in function and/or structure.

Although the present invention has been described with reference to specific exemplary embodiments, it will be recognized that the invention is not limited to the embodiments described but can be practiced with modification and alteration within the spirit and scope of the appended claims. Accordingly, the specification and drawings are to be regarded in an illustrative sense rather than a restrictive sense.

What is claimed is:

1. A method implemented by a mobile application operating on a user device, the method comprising:
obtaining, from a body scale communicatively coupled to the user device, data including biometric information of a user as measured by the body scale;
causing an imaging device communicatively coupled to the user device to capture an image of the user;
associating the biometric information of the user with the image of the user, and with a timestamp;
storing, in a memory of the user device, the biometric information of the user, the image of the user, and the timestamp; and
causing to display, based on a plurality of stored timestamps, data representing a plurality of measured pieces of biometric information of the user, along with captured images of the user, across a timeline having multiple time points.

2. The method of claim 1, further comprising:
identifying a user profile from the obtained data including the piece of biometric information of the user,
wherein said data representing the piece of biometric information of the user, the image of the user, and the generated association is stored under the user profile.

3. The method of claim 1, wherein causing the imaging device to capture the image of the user is responsive to obtaining the set of biometric information from the scale.

4. The method of claim 1, further comprising:
sending the acquired set of biometric information and the image associated with the first user profile and the timestamp to a network-accessible server system.

5. The method of claim 4, further comprising:
receiving the set of biometric information from the scale via a first communication interface, wherein the set of biometric information is sent to the network-accessible server system via a second communication interface with a greater wireless communication range than that of the first communication interface.

6. The method of claim 1, further comprising:
sending a request to capture multiple images to multiple imaging devices communicatively coupled to the user device, the multiple imaging devices configured to capture images of the user from various perspectives;
receiving multiple images from the multiple imaging devices; and
combining the multiple images received from the multiple imaging devices to generate a three-dimensional illustration of the user.

7. The method of claim 1, further comprising:
responsive to determining that the user device is within a wireless communication range of the scale, sending a request for biometric information to the scale,
wherein the set of biometric information is obtained from the scale based on sending the request for biometric information to the scale.

8. The method of claim 1, further comprising:
obtaining a series of biometric information and images of the user with timestamps corresponding to a first time period;
arranging each of the series of biometric information and the images chronologically according to the timestamps;
generating an interactive dashboard providing a multimedia presentation of the series of biometric information and images arranged chronologically according to the timestamps.

9. The method of claim 8, further comprising:
separating the biometric information by type of biometric information, wherein each type of biometric information is arranged chronologically according to the timestamps;
generating a graphical representation of each type of biometric information included in the interactive dashboard, the graphical representation including a trendline indicative of trends for each type of biometric information during the first time period.

10. The method of claim 1, wherein said identifying the first user profile associated with the user further comprises:
comparing the set of biometric information with information relating to a series of user profiles to determine that the set of biometric information is within a threshold similarity to information related to the first user profile.

11. The method of claim 3, further comprising:
obtaining a series of biometric information and images of the user with timestamps corresponding to a first time period;
arranging each of the series of biometric information and the images chronologically according to the timestamps;
generating video depicting a time-lapse illustration of changes to a body composition of the user, the video including both a graphical representation of the series of biometric information arranged chronologically according to the timestamps and the series of images arranged chronologically according to the timestamps; and
sending the generated video to an external device that facilitates sharing of the generated video between multiple viewers.

12. A method performed by a network-accessible server system to generate a multimedia presentation of biometric information and imaging information according to timestamps, the method comprising:
obtaining a first set of biometric information captured by a network-accessible scale and a first set of imaging information captured by an imaging device, wherein acquisition of the first set of biometric information causes acquisition of the first set of imaging information by the imaging device;
associating the received first set of biometric information and the first set of imaging information with a timestamp indicative of when the first set of biometric information and the first set of imaging information was obtained;
storing the first set of biometric information and the first set of imaging information and the timestamp in a first portion of memory;
adding the first set of biometric information and the first set of imaging information into a listing of a series of biometric information and imaging information stored in the first portion of memory ordered chronologically according to the timestamp; and
generating a multi-media presentation of the series of biometric information and imaging information relating to the first user profile according to the listing of the series of biometric information and imaging information.

13. The method of claim 12, wherein the first set of biometric information and the first set of imaging information are received from a user device communicatively coupled to the network-accessible scale and the imaging device, the user device configured to cause acquisition of the imaging information from the imaging device responsive to receiving the first set of biometric information from the network-accessible scale.

14. The method of claim 12, further comprising:
identifying a first user profile corresponding to the first set of biometric information;
comparing the set of biometric information with information relating to known user profiles to determine whether the set of biometric information is within a threshold similarity to information relating to any of the known user profiles; and
determining that information relating to the first user profile is within the threshold similarity to the received set of biometric information.

15. The method of claim 14, further comprising:
determining that the set of biometric information is not within the threshold similarity to information relating to any known user profile,
wherein the set of biometric information, the set of imaging information, and the timestamp are stored in a second portion of memory associated with a new user profile responsive to determining that the set of biometric information is not within the threshold similarity to information relating to any known user profile.

16. The method of claim 12, further comprising:
retrieving the series of biometric information and imaging information from the first portion of memory that include timestamps within a first time period; and
presenting the multi-media presentation as an interactive dashboard configured to display multiple types of measured pieces of biometric information of the user across timelines, each having multiple time points.

17. The method of claim 12, further comprising:
receiving the set of biometric information captured by the network-accessible scale from a user via a first communication interface, the user device configured to receive the set of biometric information from the network-accessible scale via a second communication interface, the first communication interface including a greater wireless communication range than the second communication interface; and
receiving the set of imaging information captured by the imaging device from a network node via a third communication interface.

18. The method of claim 12, further comprising:
presenting the multimedia presentation as a video depicting a time-lapse illustration of the series of biometric information and images during a first time period according to the listing; and
sending the video to an external device facilitating access to the video by a plurality of viewers.

19. A network-accessible scale comprising:
a sensor configured to capture information relating to a body composition of a user; and
a processor configured to:
inspect the information relating to the body composition of the user to determine biometric measurements relating to the user;
cause an imaging device to capture an image of the user responsive to the sensor capturing the information relating to the body composition of the user;
associate the biometric measurements and the image with a timestamp indicative of when the sensor captured the information relating to the body composition of the user; and
send the biometric measurements and the image associated with the timestamp to a user device configured to utilize the biometric measurements and the image to generate a multimedia presentation of a series of biometric measurements and images associated with the user.

20. The network-accessible scale of claim 19, wherein the processor is further configured to:
identify a first user profile associated with the user based on the determined biometric measurements;
compare the biometric measurements with information relating to known user profiles; and
determine that information relating to the first user profile is within a threshold similarity to the biometric measurements.

21. The network-accessible scale of claim 19, wherein the sensor is further configured to:
determine that the user has stepped onto the network-accessible scale, wherein the biometric information is detected responsive to determining that the user has stepped onto the network-accessible scale.

22. The network-accessible scale of claim 19, wherein the processor is further configured to:
store the biometric measurements and image associated with the timestamp.

23. The network-accessible scale of claim 22, wherein the processor is further configured to:
receive an indication from the user device that the user device is within a wireless communication range of the network-accessible scale; and
upon receiving the indication from the user device, send stored biometric measurements and images associated with the first user profile to the user device.

24. The network-accessible scale of claim 19, wherein the processor is further configured to:
receive a series of images of the user from multiple imaging devices; and
combine the multiple images received from the multiple imaging devices to generate a three-dimensional illustration of the user.
